(19) United States
(12) Patent Application Publication
(10) Pub. No.: US 2012/0289677 A1
(43) Pub. Date: Nov. 15, 2012

(54) PROCESS FOR ALKYLATING BENZENE

(75) Inventors: Laura E. Leonard, Western Springs, IL (US); Robert Haizmann, Rolling Meadows, IL (US)

(73) Assignee: UOP, LLC, Des Plaines, IL (US)

(21) Appl. No.: 13/105,680

(22) Filed: May 11, 2011

(51) Int. Cl. C08G 63/183; C07C 63/26; C07C 15/08

(52) U.S. Cl. 528/308.3; 585/446; 585/449; 562/480

(57) ABSTRACT

One exemplary embodiment can be a process for alkylating benzene. The process can include obtaining at least a portion of a stream from a transalkylation zone, combining the at least the portion of the stream from the transalkylation zone with a fuel gas stream, and providing at least a portion of the combined stream to a benzene methylation zone. Typically, the fuel gas stream includes an effective amount of one or more alkanes for alkylating at least partially from a hydrogen purification process tail gas.

### FIELD OF THE INVENTION

The present invention generally relates to a process for alkylating benzene.

### DESCRIPTION OF THE RELATED ART

Typically, an aromatic complex can process a hydrotreated naphtha feed to produce various products such as benzene and one or more xylenes. However, it may be desirable to produce higher substituted aromatics depending, e.g., on market conditions. In addition, when producing motor fuel products, increasingly stringent environmental regulations can require lower benzene content. As a consequence, there is a demand for alternative processes for removing benzene from, e.g., gasoline. Thus, systems and processes that allow flexibility to convert benzene to other and higher valued products may be desirable.

However, existing processes can use expensive catalysts and/or reactants that can require further processing to separate undesirable by-products. Thus, it would be advantageous to provide an agent that can convert benzene to other substituted aromatics while minimizing undesirable products and/or side reactions.

One exemplary technology can methylate benzene using an alkylating agent from any suitable source. The alkylating agent can be obtained from an aromatic extraction raffinate and/or a light naphtha. In such a process, a significant portion of the raffinate and light naphtha can be converted to propane.

However, such processes have several disadvantages. It would be beneficial to identify and utilize other sources within a refinery or chemical manufacturing complex for providing the suitable alkylating agent. In addition, often only a single benzene methylation stage is utilized, which can suffer insufficient selectivity for producing the desired alkylate. As a consequence, there is a desire to provide flexibility and efficiency within an aromatic complex when utilizing refinery or chemical manufacturing streams for producing alkylates.

### SUMMARY OF THE INVENTION

One exemplary embodiment can be a process for alkylating benzene. The process can include obtaining at least a portion of a stream from a transalkylation zone, combining the at least the portion of the stream from the transalkylation zone with a fuel gas stream, and providing at least a portion of the combined stream to a benzene methylation zone. Typically, the fuel gas stream includes an effective amount of one or more alkanes for alkylating at least partially from a hydrogen purification process tail gas.

Another exemplary embodiment may be a process for alkylating benzene. The process can include providing at least a portion of a stream from a transalkylation zone to a first or a second benzene methylation zone, providing a feed including one or more C4⁺ hydrocarbons to the first benzene methylation zone, and combining at least a portion of an effluent including an effective amount of one or more alkanes for alkylating from a hydrogen purification process with at least a portion of an effluent including one or more C4⁻ hydrocarbons from the first benzene methylation zone to the second benzene methylation zone.

Yet another exemplary embodiment can be a process for alkylating benzene. Generally, the process includes providing at least a portion of a stream having one or more C3⁺ hydrocarbons from a sponge absorption zone to a benzene methylation zone. Usually, the benzene methylation zone operates at a temperature of about 250- about 700° C. and a pressure of about 100- about 21,000 kPa for producing one or more xylenes.

The embodiments provided herein can utilize at least a portion of various streams, such as a fuel gas stream from a hydrogen purification process tail gas or a raffinate stream from a transalkylation zone to provide a suitable alkylating agent for benzene. Generally, it is preferred that the alkylating agent methylates benzene to form one or more xylenes. Usually, a benzene methylation zone along with an optional sponge adsorption zone can be added to an aromatic apparatus for improving aromatic alkylation. In one exemplary embodiment, a plurality of benzene methylation zones can be utilized to further enhance selectivity.

### DEFINITIONS

As used herein, the term “zone” can refer to an area including one or more equipment items and/or one or more sub-zones. Equipment items can include one or more reactors or reactor vessels, heaters, separators, exchangers, pipes, pumps, compressors, and controllers. Additionally, an equipment item, such as a reactor or vessel, can further include one or more zones or sub-zones.

As used herein, the term “stream” can be a stream including various hydrocarbon molecules, such as straight-chain, branched, or cyclic alkanes, alkenes, alkadienes, and alkynes, and optionally other substances, such as gases, e.g., hydrogen, or impurities, such as heavy metals. The stream can also include aromatic and non-aromatic hydrocarbons. Moreover, the hydrocarbon molecules may be abbreviated C1, C2, C3 . . . Cn where “n” represents the number of carbon atoms in the hydrocarbon molecule and be further characterized by a superscript “+” or “−” symbol. In such an instance, a stream characterized, e.g., as containing C3⁻, can include hydrocarbons of three carbon atoms or less, such as one or more compounds having three carbon atoms, two carbon atoms, and/or one carbon atom. Also, the symbol “A” in conjunction with a numeral and/or a superscript plus or minus may be used below to represent one or more aromatic compounds. As an example, the abbreviation “A9” may represent one or more aromatic C9 hydrocarbons.

As used herein, the term “aromatic” can mean a group containing one or more rings of unsaturated cyclic carbon radicals where one or more of the carbon radicals can be replaced by one or more non-carbon radicals. An exemplary aromatic compound is benzene having a C6 ring containing three double bonds. Moreover, characterizing a stream or zone as “aromatic” can imply one or more different aromatic compounds.

As used herein, the term “rich” can mean an amount generally of at least about 50%, and preferably about 70%, by weight, of a compound or class of compounds in a stream.

As used herein, the term “substantially” can mean an amount generally of at least about 90%, preferably about 95%, and optimally about 99%, by weight, of a compound or class of compounds in a stream.

As used herein, the term “selectivity” can be calculated as weight percent of converted alkanes that become A7⁺ alkyl groups based on the total alkanes in a reaction feed. Similarly, selectivity of alkanes in a fuel gas, e.g., C1-C4 hydrocarbons, can be the weight percent of alkanes converted to A7⁺ alkyl groups and fuel gas compounds, such as methane and ethane.

As depicted, process flow lines in the figures can be referred to interchangeably as, e.g., lines, pipes, feeds, effluents, products, or streams.

### BRIEF DESCRIPTION OF THE DRAWINGS

FIG. 1 is a schematic depiction of an aromatic production apparatus.

FIG. 2 is a schematic depiction of another exemplary aromatic production apparatus.

### DETAILED DESCRIPTION

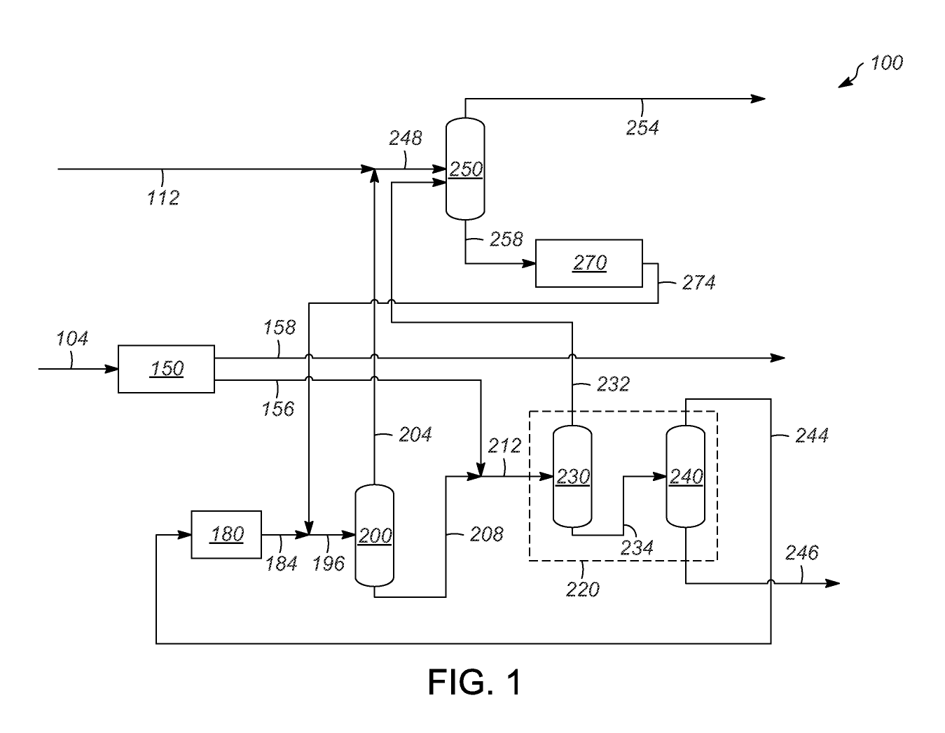

Referring to FIG. 1, an aromatic production apparatus 100 can include an extraction zone 150, a transalkylation zone 180, a stripper zone 200, a fractionation zone 220, a sponge adsorption zone 250, and a benzene methylation zone 270. Typically, the aromatic production apparatus 100 is part of a refinery or chemical manufacturing facility and produces a desired xylene, such as para-xylene or meta-xylene.

Generally, the extraction zone 150 can receive a reformate feed 104, including one or more C7⁻ hydrocarbons. The reformate feed 104 can be obtained from an overhead stream of a reformate splitter distillation column, which in turn may be obtained from a reforming zone that converts paraffins and naphthenes into one or more aromatic compounds. Typically, a reforming zone can operate at very high severity and produce about 100- about 106 research octane number gasoline reformate in order to maximize the production of one or more aromatic compounds. Generally, a hydrocarbon stream, typically a naphtha, is contacted with a reforming catalyst under reforming conditions. Such a reforming zone is disclosed in, e.g., U.S. application Ser. No. 12/689,751 filed Jan. 19, 2010.

The extraction zone 150 can utilize an extraction process, such as extractive distillation, liquid-liquid extraction or a combined liquid-liquid extraction/extractive distillation process. An exemplary extraction process is disclosed in Thomas J. Stoodt et al., “UOP Sulfolane Process”, Handbook of Petroleum Refining Processes, McGraw-Hill (Robert A. Meyers, 3rd Ed., 2004), pp. 2.13-2.23. Preferably, extractive distillation is utilized, which can include at least one column known as a main distillation column and may comprise a second column known as a recovery column.

Extractive distillation can separate components having nearly equal volatility and having nearly the same boiling point. Typically, a solvent is introduced into a main extractive-distillation column above the entry point of the hydrocarbon stream being extracted. The solvent may affect the volatility of the components of the hydrocarbon stream boiling at different temperatures to facilitate their separation. Exemplary solvents include tetrahydrothiophene 1,1-dioxide, i.e. sulfolane, n-formylmorpholine, i.e., NFM, n-methylpyrrolidinone, i.e., NMP, diethylene glycol, triethylene glycol, tetraethylene glycol, methoxy triethylene glycol, or a mixture thereof. Other glycol ethers may also be suitable solvents alone or in combination with those listed above.

The extraction zone 150 can produce a product stream 156 including one or more aromatic compounds, typically benzene and toluene, and a raffinate stream 158. Generally, the raffinate stream 158 can be sent outside the aromatic production apparatus 100 and utilized in any suitable process in the refinery or chemical manufacturing facility. In an alternative embodiment, the raffinate stream 158 may be provided to the benzene methylation zone 270.

The product stream 156 including one or more aromatics can be combined with a stripper bottom stream 208, as hereinafter described, to form a combined feed 212 to the fractionation zone 220. The fractionation zone 220 can include a benzene fractionation zone 230 and a toluene fractionation zone 240. Generally, the benzene fractionation zone 230 can include a distillation column that provides an overhead stream 232 including benzene and a bottom stream 234 including one or more A7⁺ compounds. This bottom stream 234 can be provided as a feed to the toluene fractionation zone 240, which may include a distillation column and provide an overhead stream 244 including toluene and a bottom stream 246 including one or more A8⁺ aromatics.

Usually, the bottom stream 246 can include any suitable amount of compounds, namely A8⁺ compounds that can be used to manufacture xylenes. Typically, the bottom stream 246 can be provided to a para-xylene separation zone and isomerization zone as disclosed in, e.g., U.S. Pat. No. 7,727,490, for producing desired aromatics, such as para-xylene or meta-xylene. A product stream including para-xylene may be used as a feedstock in a process to manufacture, e.g., at least one of polyethylene terephthalate and purified terephthalic acid. The overhead stream 244 can be sent to the transalkylation zone 180.

Although not wanting to be bound by any theory, at least two reactions, namely, disproportionation and transalkylation can occur in the transalkylation zone 180. The disproportionation reaction can include reacting two toluene molecules to form benzene and a xylene molecule, and the transalkylation reaction can react toluene and an aromatic C9 hydrocarbon to form two xylene molecules. As an example with respect to the transalkylation reaction, a reactant of one mole of trimethylbenzene and one mole of toluene can generate two moles of xylene, such as para-xylene, as a product. The ethyl, propyl, and higher alkyl group substituted aromatic C9-C10, can convert to lighter single-ring aromatics via dealkylation. As an example, the methylethylbenzene can lose an ethyl group through dealkylation to form toluene. Propylbenzene, butylbenzene, and diethylbenzene can be converted to benzene through dealkylation. The methyl-substituted aromatics, e.g. toluene, can further convert via disproportionation or transalkylation to benzene and xylenes. If the feed to the transalkylation zone 180 has more ethyl, propyl, and higher alkyl group substituted aromatics, more benzene can be generated in the transalkylation zone 180. Generally, the ethyl, propyl, and higher alkyl substituted aromatic compounds have a higher conversion rate than the methyl-substituted aromatic compounds, such as trimethylbenzene and tetramethylbenzene.

In the transalkylation zone 180, the overhead stream 244 may be contacted with a transalkylation catalyst under transalkylation conditions. Preferably, the catalyst is a metal stabilized transalkylation catalyst. Such a catalyst can include a solid-acid component, a metal component, and an inorganic oxide component. The solid-acid component typically is a pentasil zeolite, which may include the structures of MFI, MEL, MTW, MTT and FER (IUPAC Commission on Zeolite Nomenclature), a beta zeolite, or a mordenite. Desirably, it is a mordenite zeolite. Other suitable solid-acid components can include mazzite, NES type zeolite, EU-1, MAPO-36, MAPSO-31, SAPO-5, SAPO-11, and SAPO-41. Generally, mazzite zeolites include Zeolite Omega. Further discussion of the Zeolite Omega, and NU-87, EU-1, MAPO-36, MAPSO-31, SAPO-5, SAPO-11, and SAPO-41 zeolites is provided in, e.g., U.S. Pat. No. 7,169,368 B1.

Typically, the metal component is a noble metal or base metal. The noble metal can be a platinum-group metal of platinum, palladium, rhodium, ruthenium, osmium, or iridium. Generally, the base metal is rhenium, tin, germanium, lead, cobalt, nickel, indium, gallium, zinc, uranium, dysprosium, thallium, iron, molybdenum, tungsten, or a mixture. The base metal may be combined with another base metal, or with a noble metal. Preferably, the metal component includes rhenium. Suitable metal amounts in the transalkylation catalyst generally range from about 0.01- about 10%, preferably range from about 0.1- about 3%, and optimally range from about 0.1- about 1%, by weight. Suitable zeolite amounts in the catalyst range from about 1- about 99%, preferably from about 10- about 90%, and optimally from about 25- about 75%, by weight. The balance of the catalyst can be composed of a refractory binder or matrix that is optionally utilized to facilitate fabrication, provide strength, and reduce costs. The binder should be uniform in composition and relatively refractory. Suitable binders can include inorganic oxides, such as at least one of alumina, magnesia, zirconia, chromia, titania, boria, thoria, phosphate, zinc oxide and silica. Preferably, alumina is a binder. One exemplary transalkylation catalyst is disclosed in, e.g., U.S. Pat. No. 5,847,256.

Usually, the transalkylation zone 180 operates at a temperature of about 200- about 540° C. and a pressure of about 690- about 4,140 kPa. The transalkylation reaction can be effected over a wide range of space velocities, with higher space velocities effecting a higher ratio of para-xylene at the expense of conversion. Generally, the liquid hourly space velocity is in the range of about 0.1- about 20 hr⁻¹. The feedstock is preferably transalkylated in the vapor phase and in the presence of hydrogen. If transalkylated in the liquid phase, then the presence of hydrogen is optional. If present, free hydrogen can be associated with the feedstock and recycled hydrocarbons in an amount of about 0.1- up to about 10 moles, per mole, of an alkylaromatic.

The transalkylation zone 180 may provide a transalkylation zone effluent 184. The transalkylation zone effluent 184 can be combined with a benzene methylation zone effluent 274, as hereinafter described. In an alternative embodiment, the transalkylation zone effluent 184 may be provided to the benzene methylation zone 270. The effluents 184 and 274 can form a stripper feed 196. The stripper feed 196 is provided to the stripper zone 200.

Generally, the stripper zone 200 includes a stripper column utilizing any suitable heat source, such as a pressurized steam heat exchanger or furnace. Usually, the stripper column reboils the liquids therein to produce a stripper overhead stream 204 and a stripper bottom stream 208. Generally, the stripper overhead stream 204 can be at least a portion of the transalkylation zone effluent or stream 184 from the transalkylation zone 180. The stripper bottom stream 208 can be combined with the product stream 156 to form the combined feed 212, as described above.

The stripper overhead stream 204 can be combined with a fuel gas stream 112 to form a combined feed 248. Generally, the fuel gas stream 112 can be at least partially obtained from any suitable source, such as a hydrogen purification process, and include an effective amount of one or more alkanes. As an example, the fuel gas stream 112 can be obtained from a tail gas from a hydrogen purification unit, e.g. a pressure swing adsorber, and from the light ends produce in the transalkylation zone 180. Usually, the fuel gas stream includes one or more C3⁻ hydrocarbons and other light non-hydrocarbon gases such as hydrogen, and typically includes hydrogen, methane, ethane, ethene, and propane. The fuel gas stream 112 can include at least about 8%, preferably about 10%, by mole, one or more C3⁺ hydrocarbons, such as propane.

At least a portion of, independently, the streams 112 and 204 can form a combined feed 248 to the sponge adsorption zone 250. Also, the overhead stream 232 including benzene may also be provided to the sponge adsorption zone 250. The sponge adsorption zone 250 can remove C3 hydrocarbons, such as propane, from fuel gas using benzene. Generally, the sponge adsorption zone 250 can provide a fuel gas stream 254 having a similar composition as the fuel gas stream 112 minus one or more C3⁺ hydrocarbons, and a bottom stream providing at least a portion of a benzene methylation zone feed 258 including one or more C3 and aromatic hydrocarbons, typically benzene.

The sponge adsorption zone 250 can include a tray or packed absorber or combined packed column-tray absorber, and may be operated at a preferred temperature of about 6- about 100° C., more preferably about 10- about 20° C.; and at a pressure of about 0- about 5,000 kPa, preferably about 1,000- about 3,000 kPa. Typically, the absorber operates in a gas phase and may have from about 5- about 150 distillation trays. Distillation trays can be valve, sieve, or multiple-downcomer. The absorber can also be designed with either a random or structured packing A number of distillation stages provided by the packing can range from about 4- about 75. The absorber may be constructed from any suitable material, such as normal carbon steel, and the absorber trays can be constructed from either carbon or stainless steel. If a packing is used, it can be either carbon or stainless steel, as disclosed in, e.g., U.S. Pat. No. 7,238,843 B2.

The benzene methylation zone feed 258 can be provided to the benzene methylation zone 270. The benzene methylation zone 270, such as an alkyl, preferably methyl, can operate under any suitable conditions in the liquid or gas phase. Particularly, the reaction zone can operate at a temperature of about 250- about 700° C., preferably about 350- about 550° C.; a pressure of about 100- about 21,000 kPa, preferably about 1,900- about 3,500 kPa; a weight hourly space velocity (WHSV) of about 0.1- about 100 hr⁻¹, preferably about 2- about 10 hr⁻¹; and a hydrogen:hydrocarbon mole ratio of about 0.1:1- about 5:1, preferably about 0.5:1- about 4:1. Sufficient hydrogen may be present in the fuel gas stream 112, or additional make-up hydrogen may be provided. The reaction may occur in a gas phase to facilitate the cracking of non-aromatic hydrocarbons.

Although not wanting to be bound by theory, it is believed that the non-aromatic hydrocarbons and/or saturated groups will form methyl groups instead of alkyl groups. However, it should be understood that at least some alkylation may be occurring where groups such as, e.g., ethyl, propyl, butyl, and higher groups, can be substituted to the one or more aromatic compounds. In an exemplary embodiment, the C3 hydrocarbon conversion can be about 70%, by weight, per pass. Of the converted C3 hydrocarbon about 30%, by weight, may be converted to the desired product (A7⁺ alkyl group) while the remainder can be converted to fuel gas, typically C1 and C2 hydrocarbons. Preferably, the conversion of a substantial portion of the C3 hydrocarbons to fuel gas does not degrade the value of the stream because typically the feed is a fuel gas stream as opposed to a petrochemical grade propane. Thus, the functional selectivity of the alkylating agent to A7⁺ aromatics is typically about 100%, by weight, even when a substantial portion of the C3 hydrocarbon is converted to a lighter product. The unconverted hydrocarbon C3 can be recycled back to the benzene methylation zone 270 via the transalkylation zone 180. Once through hydrogen is preferred due to the high methane in recycled hydrogen. Alternatively, a recycle gas can be purified by any acceptable means such as but not limited to pressure swing adsorption or a membrane.

Any suitable catalyst may be utilized such as at least one molecular sieve including any suitable material, e.g., alumino-silicate. The catalyst can include an effective amount of the molecular sieve, which can be a zeolite with at least one pore having a 10 or higher member ring structure and can have one or higher dimension. Typically, the zeolite can have a Si/Al₂ mole ratio of greater than about 10:1, preferably about 20:1- about 60:1. Preferred molecular sieves can include BEA, MTW, FAU (including zeolite Y in both cubic and hexagonal forms, and zeolite X), MOR, LTL, ITH, ITW, MEL, FER, TON, MFS, IWW, MFI, EUO, MTT, HEU, CHA, ERI, MWW, and LTA. Preferably, the zeolite can be MFI and/or MTW. Suitable zeolite amounts in the catalyst may range from about 1- about 99%, and preferably from about 10- about 90%, by weight. The balance of the catalyst can be composed of a refractory binder or matrix that is optionally utilized to facilitate fabrication, provide strength, and reduce costs. Suitable binders can include inorganic oxides, such as at least one of alumina, magnesia, zirconia, chromia, titania, boria, thoria, phosphate, zinc oxide, and silica.

Generally, the catalyst is essentially absent of at least one metal, and typically includes less than about 0.1%, by weight, of total metal based on the weight of the catalyst. Moreover, the catalyst preferably has less than about 0.01%, more preferably has less than about 0.001%, and optimally has less than about 0.0001%, by weight, of total metal based on the weight of the catalyst. Generally, the benzene methylation zone 270 can provide the benzene methylation zone effluent 274, which can be utilized as a part of the stripper feed 196, as discussed above.

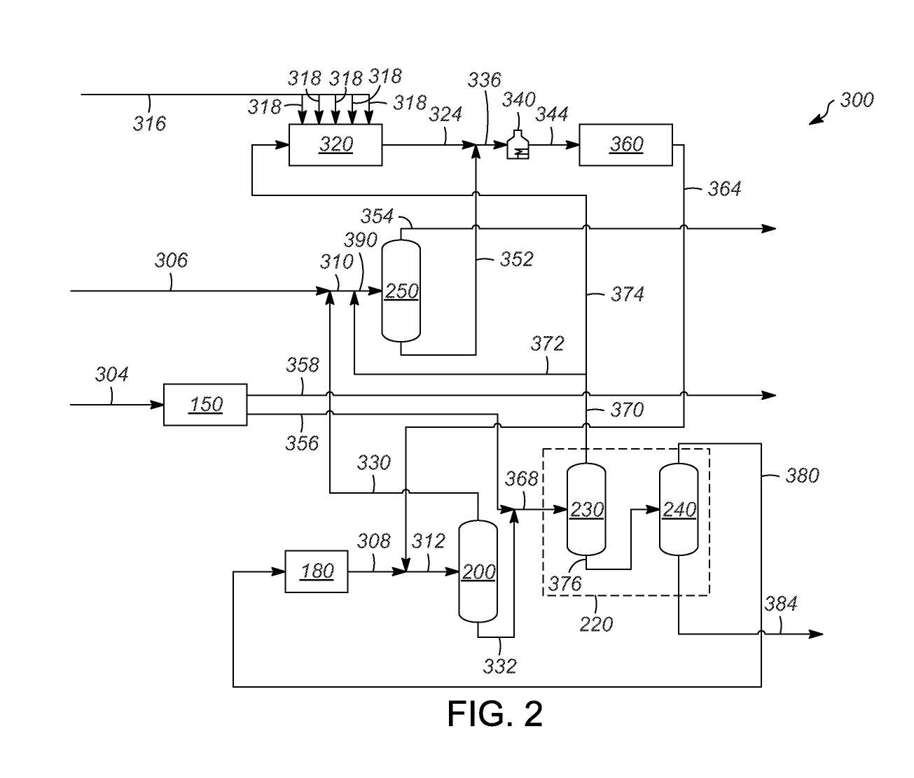

Referring to FIG. 2, another exemplary aromatic production apparatus 300 is depicted, and can be utilized in a similar facility and produce similar products as the aromatic production apparatus 100. The aromatic production apparatus 300 can include the extraction zone 150, the transalkylation zone 180, the stripper zone 200, the fractionation zone 220, and the sponge adsorption zone 250. Generally, these zones are similar to those described above. In addition, the streams flowing to and from these zones can be substantially similar as the streams described above. In addition, the aromatic production apparatus 300 can also include a first benzene methylation zone 320 and a second benzene methylation zone 360.

Generally, a reformate feed 304 can be provided to the extraction zone 150. The extraction zone 150 can provide a product stream 356 and a raffinate stream 358. The raffinate stream 358 can exit the aromatic production apparatus 300 and be used elsewhere in the refinery or chemical manufacturing facility. Optionally, at least a portion of the raffinate stream 358 can be directed to the first benzene methylation zone 320.

The product stream 356 can be combined with a stripper bottom stream 332, as hereinafter described, and form a feed 368 to the fractionation zone 220. The fractionation zone 220 can include the benzene fractionation zone 230 and the toluene fractionation zone 240.

The feed 368 can be provided to the benzene fractionation zone 230, which in turn provides an overhead stream 370 including benzene, which can be split into streams 372 and 374 as hereinafter described, and a bottom stream 376 including one or more A7⁺ hydrocarbons. The bottom stream 376 can be provided to the toluene fractionation zone 240. The toluene fractionation zone 240 can provide a bottom stream 384 including one or more A8⁺ aromatics. The bottom stream 384 can be provided to any suitable zone, such as a para-xylene separation zone and an isomerization zone for obtaining one or more desired products, as described above. The overhead stream 380, including toluene, can be provided to the transalkylation zone 180.

Generally, the transalkylation zone 180, as described above, can provide a transalkylation zone effluent 308. The transalkylation zone effluent 308 can be combined with the second benzene methylation zone effluent 364, as hereinafter described, to form a stripper feed 312. In an alternative embodiment, the transalkylation zone effluent 308 may be provided to the first benzene methylation zone 320 and/or second benzene methylation zone 360.

The stripper feed 312 can be provided to the stripper zone 200. The stripper zone 200 can provide an overhead stream 330 and stripper bottom stream 332. This overhead stream 330 may include one or more C3⁺ hydrocarbons due to dealkylation of one or more C9⁺ aromatics. The stripper bottom stream 332 can be combined with the product stream 356 to form the feed 368.

The overhead stream 330 can be combined with a fuel gas stream 306. Generally, the fuel gas stream 306 can have the same composition as the fuel gas stream 112, as described above. The streams 306 and 330 can form a combined stream 310, which in turn can be combined with benzene from a stream 372 split from the overhead stream 370. Thus, the sponge adsorption zone 250 can include at least a portion of, independently, the overhead stream 330, the fuel gas stream 306, and the stream 372 from a fractionation zone 220. The streams 310 and 372 can in turn, be combined to form a feed 390 for the sponge adsorption zone 250, as described above. Alternatively, the streams 310 and 372 can be provided to and mixed in the sponge adsorption zone 250.

The sponge adsorption zone 250 can provide a bottom stream 352 and an overhead stream 354, which can include a fuel gas having a composition substantially the same as the fuel gas stream 254, as described above. Alternatively, the sponge adsorption zone 250 can be omitted and the feed 390 can be combined with the first benzene methylation zone effluent 324, as described below.

Another portion 374 of the overhead stream 370 can be provided to a first benzene methylation zone 320, operating similarly as the benzene methylation zone 270 described above. In addition to receiving the portion 374, the first benzene methylation zone 320 can also receive a C5 naphtha stream 316, including one or more C5 hydrocarbons. Usually, one or more C5-C6 hydrocarbons are provided with benzene to the first benzene methylation zone 320. Pentane can be provided from naphtha and/or a depentanizer overhead stream. Optionally, the C5 naphtha stream 316 may be split into multiple feed streams 318 and provided at multiple feed points into the first benzene methylation zone 320. The C5 multipoint injection of C5 hydrocarbons in the first benzene methylation zone 320 can maintain high benzene and pentane ratios. The first benzene methylation zone 320 often includes a single reactor. Alternatively, the stream 358 may optionally be combined with the stream 316 and provided as a feed to the first benzene methylation zone 320.

Generally, the first benzene methylation zone 320 can provide a first benzene methylation zone effluent 324 that can be combined with the sponge adsorption zone bottom stream 352, which can include propane and benzene. The streams 352 and 324 can form a combined feed 336, which is passed through a heater 340, which can be any suitable heating device, such as a furnace.

After being heated, a feed 344 can be provided to the second benzene methylation zone 360 operating similarly as the benzene methylation zone 270, as described above. Often, the second benzene methylation zone 360 can provide a second benzene methylation zone effluent 364, which can be combined with the transalkylation zone effluent 308, as described above.

Generally, the second benzene methylation zone 360 operates at a temperature of about 10- about 100° C., preferably about 20- about 80° C. higher than the first benzene methylation zone 320. Typically, the first benzene methylation zone 320 operates at less severe conditions using heavier hydrocarbons such as one or more C4⁺ alkanes. Additional one or more C3 hydrocarbons can be provided to the first benzene methylation zone effluent 324 and fed to the second benzene methylation zone 360. Although not wanting to be bound by theory, a portion of the one or more C4⁺ alkanes can be converted to lighter one or more C4⁻ alkanes. The first benzene methylation zone effluent 324 may be provided to the second benzene methylation zone 360 at higher severity where C4⁻ alkanes may be more reactive. Optionally, the first benzene methylation zone effluent 324 containing C3 hydrocarbons can be alkylated with fresh propane in the second benzene methylation zone 360 at an elevated temperature.

The first benzene methylation zone 320 can be operated to achieve about 20- about 45%, by weight, benzene conversion to one or more A7⁺ hydrocarbons, and about 60- about 100%, by weight, one or more C4⁺ hydrocarbons conversion. Generally, the one or more C4⁺ alkanes can be converted to one or more A7⁺ alkyl groups, C4⁻ hydrocarbons, and C2⁻ hydrocarbons. Usually, the one or more C4⁺ alkanes selectivity to the one or more A7⁺ aromatics is at least about 20%, by weight, preferably about 30%, by weight. Typically, the one or more C4⁺ hydrocarbons selectivity to the one or more C3 hydrocarbons are about 25- about 50%, by weight. The second benzene methylation zone 360 can be at an elevated temperature with a benzene conversion of at least about 20%, by weight, preferably at least about 30%, by weight, conversion per pass. Usually at these conditions about 30- about 40%, by weight, of the C3 hydrocarbons converted are in the form of one or more A7⁺ alkyl groups in the product. The overall selectivity to one or more A7⁺ alkyl groups for the one or more C4⁺ hydrocarbons may be about 30- about 50%, by weight, for zones 320 and 360. Generally, the one or more C4⁺ hydrocarbons conversion to one or more A7⁺ alkyl groups is higher utilizing the two stages in combination as disclosed herein when compared to the performance that can be achieved using only the first benzene methylation zone 320.

As an example, the embodiments disclosed herein can achieve about 10- about 50%, by weight, increase in a xylene yield from the aromatic production apparatus 100 by utilizing fuel gas and one or more C4⁺ alkanes. The two zones 320 and 360 may have C4⁺ hydrocarbon stream, such as a higher raffinate or a light naphtha, selectivity to one or more A7⁺ alkyl groups processing a light naphtha or raffinate only in a first benzene methylation zone 320 without subsequent conversion of propane in the second benzene methylation zone 360. Therefore, the overall selectivity of alkylation can increase from about 25%, by weight, with a first benzene methylation zone 320 and about 40%, by weight, with a second benzene methylation zone 360.

In an alternative embodiment, the sponge adsorption zone 250 can be positioned upstream of the first benzene methylation zone 320. The conditions of the first benzene methylation zone 320 may be tailored so that much of the one or more C3 hydrocarbons passes through largely unreacted with substantial conversion of the C4⁺ hydrocarbons. The effluent from the first benzene methylation zone 320 is provided to the second benzene methylation zone 360, which can operate at higher severity, i.e. higher temperature and/or lower pressure, to convert the one or more C4⁻ hydrocarbons. Although not wanting to be bound by theory, the selectivity of the alkylating agent can be maximized in the alkylation reaction with benzene and cracking of C1 or C2 hydrocarbons may be minimized.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing, all temperatures are set forth in degrees Celsius and, all parts and percentages are by weight, unless otherwise indicated.

From the foregoing description, one skilled in the art can easily ascertain the essential characteristics of this invention and, without departing from the spirit and scope thereof, can make various changes and modifications of the invention to adapt it to various usages and conditions.

1. A process for alkylating benzene, comprising:
A) obtaining at least a portion of a stream from a transalkylation zone;
B) combining the at least the portion of the stream from the transalkylation zone with a fuel gas stream comprising an effective amount of one or more alkanes for alkylating at least partially from a hydrogen purification process tail gas; and
C) providing at least a portion of the combined stream to a benzene methylation zone.

2. The process according to claim 1, further comprising a sponge absorption zone receiving the combined stream and a stream comprising benzene, and providing an effluent to the benzene methylation zone.

3. The process according to claim 1, further comprising obtaining a stream comprising benzene from a fractionation zone.

4. The process according to claim 1, further comprising providing an effluent from the transalkylation zone to a stripper zone.

5. The process according to claim 4, wherein the at least the portion of the stream from the transalkylation zone is obtained from the stripper zone.

6. The process according to claim 1, wherein the fuel gas stream comprises at least about 8%, by mole, of one or more C3⁺ hydrocarbons.

7. The process according to claim 3, providing a product stream comprising one or more aromatics from an extraction zone to the fractionation zone.

8. The process according to claim 7, wherein the extraction zone uses a solvent comprising at least one of tetrahydrothiophene 1,1-dioxide, n-formylmorpholine, n-methylpyrrolidinone, diethylene glycol, triethylene glycol, tetraethylene glycol, methoxy triethylene glycol, or a mixture thereof.

9. The process according to claim 3, wherein the fractionation zone provides a stream comprising toluene.

10. The process according to claim 1, wherein the benzene methylation zone operates at a temperature of about 250- about 700° C., a pressure of about 100- about 21,000 kPa, and a hydrogen:hydrocarbon mole ratio of about 0.1:1- about 5:1.

11. The process according to claim 10, wherein the benzene methylation zone comprises a catalyst, wherein the catalyst comprises a molecular sieve.

12. The process according to claim 7, wherein a bottom stream from the fractionation zone comprises para-xylene; and further processing the product stream for manufacturing at least one of polyethylene terephthalate and purified terephthalic acid.

13. A process for alkylating benzene, comprising:
A) providing at least a portion of a stream from a transalkylation zone to a first or a second benzene methylation zone;
B) providing a feed comprising one or more C4⁺ hydrocarbons to the first benzene methylation zone; and
C) combining at least a portion of an effluent comprising an effective amount of one or more alkanes for alkylating from a hydrogen purification process with at least a portion of an effluent comprising one or more C4⁻ hydrocarbons from the first benzene methylation zone to the second benzene methylation zone.

14. The process according to claim 13, wherein the second benzene methylation zone operates at a temperature of about 10- about 100° C. higher than the first benzene methylation zone.

15. The process according to claim 13, further comprising providing an effluent from the transalkylation zone to a stripper zone, and in turn, providing an overhead stream from the stripper zone to a sponge absorption zone.

16. The process according to claim 15, further comprising combining the overhead stream and a fuel gas stream comprising the effective amount of one or more alkanes for alkylating from a hydrogen purification process tail gas.

17. The process according to claim 16, wherein the fuel gas stream comprises at least about 8%, by mole, one or more C3⁺ hydrocarbons.

18. The process according to claim 16, wherein the fuel gas stream is obtained from a pressure swing adsorber.

19. The process according to claim 13, wherein the first and second benzene methylation zones operate, independently, at a temperature of about 250- about 700° C., a pressure of about 100- about 21,000 kPa, and a hydrogen:hydrocarbon mole ratio of about 0.1:1- about 5:1.

20. A process for alkylating benzene, comprising:
providing at least a portion of a stream comprising one or more C3⁺ hydrocarbons from a sponge absorption zone to a benzene methylation zone wherein the benzene methylation zone operates at a temperature of about 250- about 700° C. and a pressure of about 100- about 21,000 kPa for producing one or more xylenes.
